(19) United States
(12) Patent Application Publication
(10) Pub. No.: US 2023/0407275 A1
(43) Pub. Date: Dec. 21, 2023

(54) ENHANCEMENT OF PREDICTABLE AND TEMPLATE-FREE GENE EDITING BY THE ASSOCIATION OF CAS WITH DNA POLYMERASE

(71) Applicant: New York University, New York, NY (US)

(72) Inventors: Chengzu LONG, New York, NY (US); Qiaoyan YANG, New York, NY (US)

(21) Appl. No.: 18/251,384

(22) PCT Filed: Nov. 4, 2021
(86) PCT No.: PCT/US2021/058135

§ 371 Date: May 1, 2023

Related U.S. Application Data
(60) Provisional application No. 63/109,909, filed on Nov. 5, 2020.

(51) Int. Cl. C12N 9/12; C07K 14/005; C12N 9/22; C12N 15/63; C12N 15/10

(52) U.S. Cl. CPC C12N 9/1252; C07K 14/005; C12N 9/22; C12N 15/63; C12N 15/102; C07K 2319/09; C12N 2310/20

(57) ABSTRACT

Provided are compositions and methods for precise genome editing. The compositions include a fusion protein comprising a T4 DNA polymerase segment and a segment of an MS2 bacteriophage coat protein. The fusion protein operates with a Cas enzyme and one or more guide RNAs to produce one or more indels. The indel is produced in a DNA repair template free manner. Methods for producing the indels are also provided. A method includes introducing into the cell a fusion protein containing a T4 DNA polymerase segment and a segment of an MS2 bacteriophage coat protein, a Cas enzyme, and a guide RNA comprising MS2 protein binding sites. The guide RNA directs the Cas enzyme, the T4 DNA polymerase and the MS2 binding protein to the selected chromosome locus to produce the indel. The indel may correct a mutation in an open reading frame encoded by the selected chromosome locus.

### CROSS-REFERENCE TO RELATED APPLICATIONS

This application claims priority to U.S. provisional application No. 63/109,909, filed Nov. 5, 2020, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Nov. 3, 2021, is titled “SpCas9_ST25.txt” and is 29,207 bytes in size.

### BACKGROUND

Clustered regularly interspaced short palindromic repeats (CRISPR)/CRISPR-associated proteins (Cas)-based genome editing has emerged as one of the most powerful tools for sequence-specific gene editing. However, common gene editing strategies often require homology directed repair mediated knock-ins, a method which can be inefficient or infeasible such as in the post-mitotic cells of the central nervous system and heart, or more recently, base editing approaches, which cannot address diseases caused by insertions and deletions (indels). Recently multiple groups demonstrated that SpCas9-mediated template-free nucleotide insertions are precise and predictable. However, there remains an ongoing and unmet need for improved compositions and methods for precisely generating indels for a variety of purposes. The present disclosure is pertinent to this need.

### BRIEF SUMMARY

The present disclosure provides compositions and methods for precise genome editing. The compositions include a fusion protein comprising a T4 DNA polymerase segment and a segment of an MS2 bacteriophage coat protein. The fusion protein operates with a Cas enzyme and one or more guide RNAs to produce one or more indels. In embodiments, the indel is produced using non-homologous end joining (NHEJ), which is at least in part facilitated by the T4 DNA polymerase that is a component of a genome editing system encompassed by the disclosure. The disclosure thereby provides for producing an indel in a DNA repair template free manner. The fusion protein functions as a component of a CRISPR system in the nucleus of the cell. Accordingly, any protein described herein may include at least one nuclear localization signal. The fusion protein may also include one or more linkers that separate, for example, the T4 DNA polymerase and the MS2, and/or that separate a segment of the fusion protein from the nuclear localization signal. In embodiments, the fusion protein comprises a self-cleaving peptide sequence, which can, for example, promote ribosomal skipping during translation. Thus, the fusion protein may be encoded by an mRNA that encodes additional amino acids on the N- or C-terminal ends of the fusion protein which, by operation of a self-cleaving peptide sequence, are not translated as a part of a contiguous polypeptide that comprises the T4 DNA polymerase and the MS2 protein segment.

In an aspect, the disclosure comprises a complex comprising a Cas enzyme, a guide RNA comprising MS2 bacteriophage coat protein binding sites, a protein comprising a T4 DNA polymerase, and an MS2 binding protein. The complex may further comprise a guide RNA comprising MS2 protein binding sequences. Cells comprising a described fusion protein and a described complex are also included. Pharmaceutical compositions comprising the described fusion proteins are also provided. Such compositions may also comprise a guide RNA and a Cas enzyme. Cells comprising the described fusion proteins and complexes are also included. The disclosure also provides expression vectors and cDNAs encoding the described fusion proteins, as well as kits comprising the same and/or additional components.

In another aspect, the disclosure provides a method for producing an indel at a selected chromosome locus in a cell. The method comprises introducing into the cell a described fusion protein, a Cas enzyme, and a guide RNA comprising MS2 protein binding sites, wherein the guide RNA directs the Cas enzyme, the T4 DNA polymerase and the MS2 binding protein to the selected chromosome locus, to thereby produce the indel. In embodiments, the indel corrects a mutation in an open reading frame encoded by the selected chromosome locus, or converts a sequence into an open reading frame. In embodiments, the selected chromosome locus comprises a mutation in a gene that is correlated with a monogenic disease. In one non-limiting embodiment, the monogenic disease is muscular dystrophy, and wherein the selected chromosome locus includes a gene that includes a mutated dystrophin protein. Thus, in an embodiment, the indel corrects the gene encoding the mutated dystrophin protein. In certain examples, the indel comprises a one or two base pair insertion.

### BRIEF DESCRIPTION OF THE FIGURES

FIGS. 1A-H. CRISPR/Cas9-guided T4 DNA polymerase facilitates the generation of insertions via filling in the staggered DNA with 5′ overhang. FIG. 1A. Schematic showing the repair processes and outcomes of Cas9-induced DSBs. DNA polymerases enable to fill in the 5′-single base overhangs created by Cas9, thus, facilitating the production of 1-bp insertions. Exonucleases promote end resection at Cas9-induced DSB ends, eventually favoring the generation of deletions. FIG. 1B. Illustration of tdTomato reporter plasmids containing a deletion of adenosine at position 151 (del151A) and sequences of the guide RNA. The cutting sites of SpCas9 are shown by arrowheads. The sequence of nucleotide sequent for Del151A is SEQ ID NO:1. The sequence for the WT sequence is SEQ ID NO:2. The sequence of the top strand of tdTomato-sgRNA and PAM is SEQ ID NO:3. The sequence of the bottom strand of tdTomato-sgRNA and PAM is SEQ ID NO4. FIG. 1C. Architecture of DNA polymerase-expressing vectors. EF1A, promoter of elongation factor 1-alpha; NLS, nuclear localization signal; MS2, MS2 bacteriophage coat protein. FIGS. 1D-1E. Cas9-induced insertions profiles and frequencies of tdTomato del151A site in tdTomato⁺/EGFP⁺ populations (D) and tdTomato⁻/EGFP⁺ populations (E). Different cell populations were sorted from tdTomato del151A reporter cells transfected with Cas9 or co-transfected with Cas9 and MS2-tagged DNA polymerases. Target regions were amplified and sequenced by Sanger sequencing. All the sequencing files were analyzed via Synthego ICE software tool. The arrowheads point to 2-bp insertion that was significantly increased in T4 DNA polymerase-expression cells relative to cells with other treatments. FIG. 1F. Indels profiles and frequencies produced in tdTomato reporter cells transfected with Cas9 or co-transfected with Cas9 and T4 DNA polymerase. Target regions were amplified and sequenced by deep sequencing. FIG. 1G. The pattern of 1-bp, 2-bp and 3-bp insertion in control (Cas9 only) and T4 DNA polymerase with Cas9 co-transfection cells. FIG. 1H. Indels profiles and frequencies of three endogenous genome sites (Mybpc3-323-g3, LMNA-Ex3-g2, Mybpc3-323-g2) in 293T cells induced by Cas9 or CasPlus (+T4 Pol). The sequence of the Mybpc3-323-g3 (PAM) is SEQ ID NO:5. The sequence of the LMNA-Ex3-g2 (PAM) is SEQ ID NO:6. The sequence of the Mybpc3-323-g2 (PAM) is SEQ ID NO:7.

FIGS. 2A-2G. CRISPR/Cas9-guided T4 DNA polymerase impairs MMEJ repair pathway. FIG. 2A. Schematic showing the MMEJ process and outcome after Cas9 cleavage in the presence of T4 DNA polymerase. At the DSB ends, MS2-tagged T4 DNA polymerase inhibits relatively long-range end resection via filling in the gaps created by exonucleases, therefore, leading to the products with small deletions or insertions. FIGS. 2B-2G show indel profiles and frequencies at six endogenous genome sites in 293T cells induced by Cas9 (CTR) or CasPlus (T4 Pol). In B, Target site 1: DMD-Ex51-g5 (PAM) is SEQ ID NO:8. In C, the sequence of Target site 2: LMNA-Ex2-g2 (PAM) is SEQ ID NO:9. In D, the sequence of Target site 3: LMNA-Ex2-g1 (PAM) is SEQ ID NO:10. In E, Target site 4: DMD-Ex43-g1 (PAM) is SEQ ID NO:11. In F, the sequence of Target site 5: DMD-Ex51-g1 (PAM) is SEQ ID NO:12. In G, the sequence of Target site 6: DMD-Ex51-g2 (PAM) is SEQ ID NO:13.

FIG. 3A. Vectors for expression of Cas9-DNA polymerase fusion proteins. Cbh, cytomegalovirus (CMV) and chicken β-actin hybrid promoter.

FIG. 3B. Indels profiles and frequencies in tdTomato del151A cell lines overexpressed with SpCas9, SpCas9-linker-Pollambda, SpCas9-linker-Polmu, SpCas9-linker-Polbeta, SpCas9-linker-Pol4 or SpCas9-linker-T4 DNA Pol. No significant difference was detected among all the treatments.

FIG. 4. Illustration of interaction between MS2 and T4 proteins, Cas9, and a single guide RNA (sgRNA) with MS2 sgRNA binding structures, cleavage by Cas9, and T4 fill-in and ligation to produce a +1 bp insertion.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein by reference as they exist in the database on the filing date of this application or patent.

In embodiments, the disclosure provides a T4 DNA polymerase/Cas9 system, referred to herein as “CasPlus”, to precisely model and correct mutations by producing predictable indels formed following Cas9 cleavage. In one embodiment the Cas9 is derived from Streptococcus pyogenes (“SpCas9”). The system creates indels in a DNA repair template free manner. In embodiments, the indel is produced using NHEJ which is at least in part facilitated by the T4 DNA polymerase that is a component of the system.

By designing the described CasPlus system with an enhanced probability of generating preferred indels, the disclosure includes generation of isogenic patient cells with greater efficiency as compared to traditional HDR methods. The presently provided results demonstrate the utility of CasPlus system with designed gRNAs for traits beyond cleavage efficiency and gene specificity and the capacity to harness predictable indel formation for modeling and correction of a wide-range of indel-based diseases. Thus, the present disclosure provides compositions and methods for producing precise insertion and/or deletions in a guide RNA targeted segment of a chromosome. Accordingly, the disclosure in certain embodiments is used to produce indels. Indels comprise an insertion or deletion of 1, 2, 3, 4, or 5, nucleotides, with concomitant changes on the complementary strand, thus resulting in an insertion or deletion of 1-10 base pairs (bp), inclusive. The indel may comprise any desired change by using one or more suitable guide RNAs in conjunction with the protein complexes as further described herein.

In non-limiting embodiments, the indel is produced within a protein coding segment of a chromosome, at a splice junction, in a promoter, in an enhancer element, or at any other location wherein generation of an indel is desirable, provided a suitable proto adjacent motif (PAM) is proximal to the location of the indel. In embodiments, the indel corrects a mutation that is associated with a condition or disorder. In embodiments, the indel corrects a frameshift mutation, a missense mutation, or a nonsense mutation. In embodiments, the indel changes a codon for at least one amino acid in a protein coding sequence, and thus may correct a mutation in an exon to a normal (e.g., non-disease associated) exon. In embodiments, a homozygous indel may be produced. In embodiments, the indel corrects a deleterious mutation that is a component of a monogenic disorder, e.g., a disorder caused by variation in a single gene. In embodiments, the monogenic disorder is an X-linked disorder. In non-limiting embodiments, the monogenic disorder is any of sickle cell anemia, cystic fibrosis, Huntington disease, Tay-Sachs disease, phenylketonuria, mucopolysaccharidoses, lysosomal acid lipase deficiency, glycogen storage diseases, galactosemia, Hemophilia A, Rett's syndrome, or any form of muscular dystrophy, such as Duchenne muscular dystrophy (DMD). In a non-limiting embodiment, the indel corrects a mutation in the human dystrophin gene. In embodiments, the indel corrects a mutation (including but not necessarily limited to a deletion) in the human dystrophin gene that is comprised by one or more human dystrophin gene exons 2-10 or 45-55, each inclusive. In embodiments, the indel corrects one or more out-frame mutations within exons by producing a single base pair insertion. Thus, the disclosure includes exon reshaping, such as reframing an out of frame reading frame. In embodiments, the indel restores functional dystrophin expression in cells in which the mutation is corrected. In non-limiting embodiments, the disclosure provides for introducing a 1 bp insertion in human dystrophin gene exon 43, 45, 49, or 51. The amino acid sequence of human dystrophin and the sequence of the gene encoding human dystrophin is known in the art, such as via NCBI Gene ID: 1756, including all accession numbers therein, and in NCBI accession number NG_012232.

In embodiments, the disclosure provides fusion proteins that facilitate the association of T4 DNA polymerase with a Cas nuclease. In embodiments, the fusion proteins comprise an MS2 domain and a T4 DNA polymerase domain, representative sequences of which are described herein.

In embodiments, the disclosure provides for more frequent indel production relative to a control. In embodiments, the control comprises a an indel production value obtained by using an MS2 protein fused to a DNA polymerase that is not a T4 DNA polymerase, or a protein that does not exhibit nuclease activity, such as a detectable protein, non-limiting examples of which are provided herein and comprise Green Fluorescent Protein (GFP), but other proteins may be used, such a mCherry.

In embodiments, a fusion protein of the disclosure may comprise one or more ribosomal skipping sequences, which are also referred to in the art as “self-cleaving” amino acid sequences. These are typically about 18-22 amino acids long. Any suitable sequence can be used, non-limiting example of which include T2A, comprising the amino acid sequence: EGRGSLLTCGDVEENPGP (SEQ ID NO:14); P2A, comprising the amino acid sequence ATNFSLLKQAGDVEENPGP (SEQ ID NO:15); E2A, comprising the amino acid sequence QCTNYALLKLAGDVESNPGP (SEQ ID NO:16); and F2A, comprising the amino acid sequence VKQTLNFDLLKLAGDVESNPGP (SEQ ID NO:17).

In embodiments, the fusion proteins comprise linking amino acids (e.g., linkers) that separate one or more protein domains. The linker is typically at least two amino acids long, and may include a GS sequence, but other sequences may be used. In embodiments, the linker is from 3-100 amino acids in length. In embodiments, a linker sequences comprises or consists of a “GS” sequence. In embodiments, the linker comprises or consists of the sequence SAGGGGSGGGGSGGGGSG (SEQ ID NO:18).

In embodiments, a fusion protein of the disclosure includes one or more nuclear localization signals, representative and non-limiting examples of which are provided herein. In general, for eukaryotic purposes, a nuclear localization signal comprises one or more short sequences of positively charged lysines or arginines.

In non-limiting embodiments, the disclosure provides a fusion protein that comprise an MS2 segment and a DNA polymerase segment, which may also include the aforementioned linking amino acids, nuclear localization signals, and ribosome skipping/self-cleaving sequences. A segment means a section of the described protein that contains contiguous amino acid sequences. In embodiments, the segment is of sufficient length to retain the function of protein to participate in the described method and is thus a functional segment. In embodiments, a segment comprises a contiguous segment of a described protein that includes contiguously 80%-99% of a described amino acid sequence.

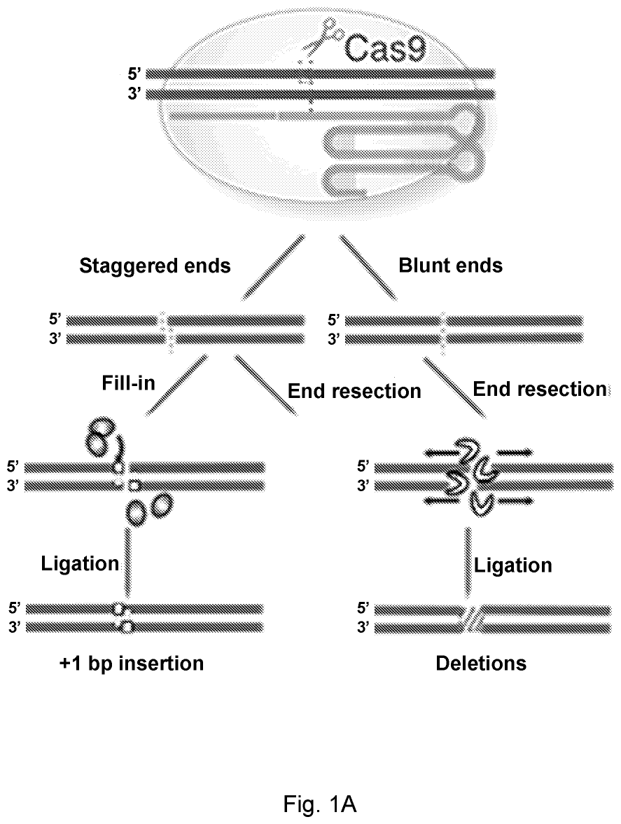

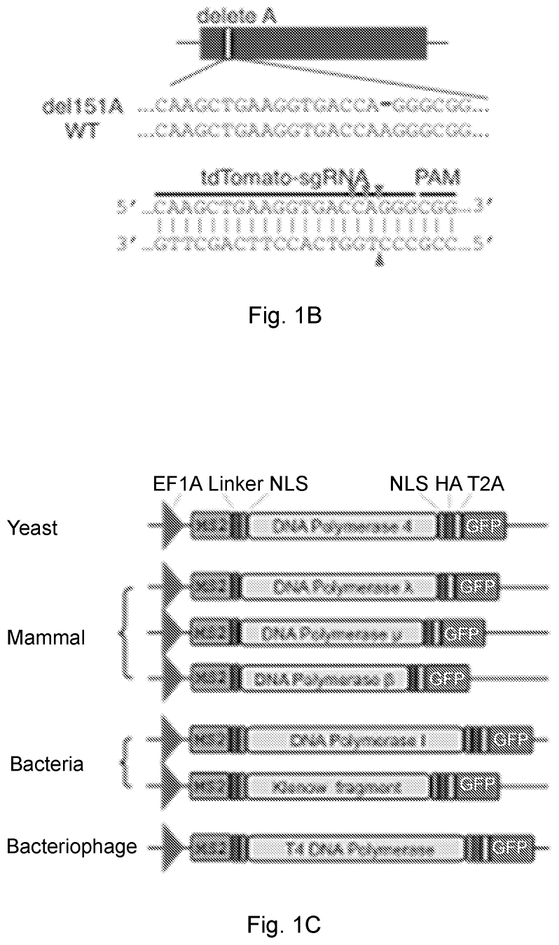

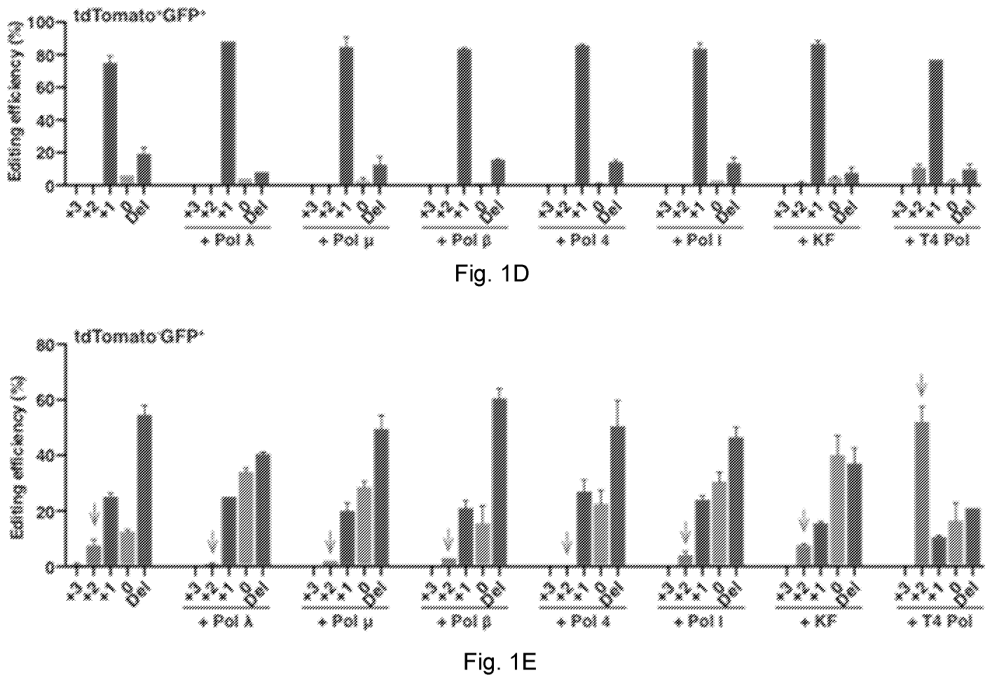

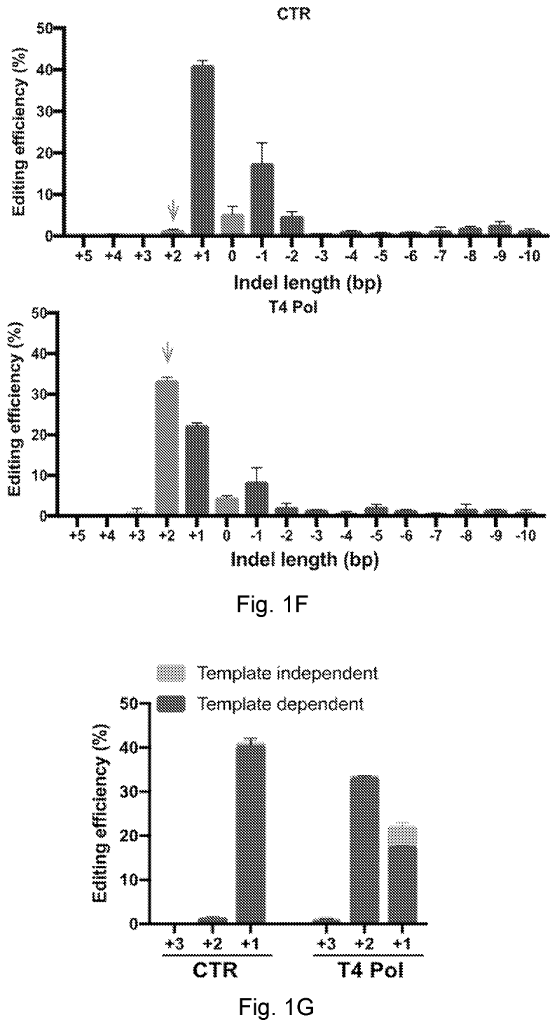

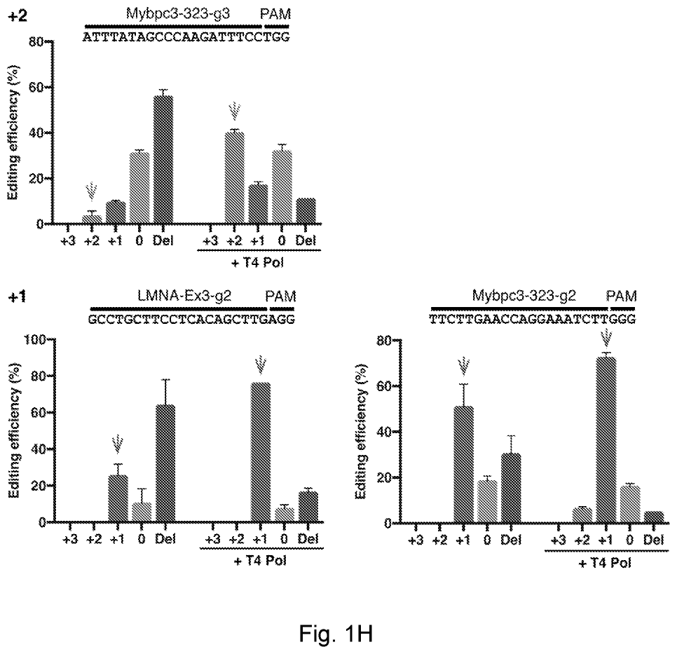

In an embodiment, the DNA polymerase is T4 DNA polymerase, but other DNA polymerases, that enable the fill in of overhang maybe used, such as T7 DNA polymerase and Rb69 DNA polymerase. We have demonstrated that the following DNA polymerases do not function in the described system: DNA polymerase lambda, DNA polymerase Mu, DNA polymerase Beta, yeast derived DNA polymerase 4, bacteria derived DNA polymerase I and Klenow fragment all do not exhibit adequate or any detectable function (see, for example, FIGS. 1D-1E).

In an embodiment, the T4 DNA polymerase comprises the sequence:

(SEQ ID NO: 19
KEFYISIETVGNNIVERYIDENGKERTREVEYLPTMFRHCKEESKYKDI
YGKNCAPQKFPSMKDARDWMKRMEDIGLEALGMNDFKLAYISDTYGSEI
VYDRKFVRVANCDIEVTGDKFPDPMKAEYEIDAITHYDSIDDRFYVFDL
LNSMYGSVSKWDAKLAAKLDCEGGDEVPQEILDRVIYMPFDNERDMLME
YINLWEQKRPAIFTGWNIEGFDVPYIMNRVKMILGERSMKRFSPIGRVK
SKLIQNMYGSKEIYSIDGVSILDYLDLYKKFAFTNLPSFSLESVAQHET
KKGKLPYDGPINKLRETNHQRYISYNIIDVESVQAIDKIRGFIDLVLSM
SYYAKMPFSGVMSPIKTWDAIIFNSLKGEHKVIPQQGSHVKQSFPGAFV
FEPKPIARRYIMSFDLTSLYPSIIRQVNISPETIRGQFKVHPIHEYIAG
TAPKPSDEYSCSPNGWMYDKHQEGIIPKEIAKVFFQRKDWKKKMFAEEM
NAEAIKKIIMKGAGSCSTKPEVERYVKFSDDFLNELSNYTESVLNSLIE
ECEKAATLANTNQLNRKILINSLYGALGNIHFRYYDLRNATAITIFGQV
GIQWIARKINEYLNKVCGTNDEDFIAAGDTDSVYVCVDKVIEKVGLDRF
KEQNDLVEFMNQFGKKKMEPMIDVAYRELCDYMNNREHLMHMDREAISC
PPLGSKGVGGFWKAKKRYALNVYDMEDKRFAEPHLKIMGMETQQSSTPK
AVQEALEESIRRILQEGEESVQEYYKNFEKEYRQLDYKVIAEVKTANDI
AKYDDKGWPGFKCPFHIRGVLTYRRAVSGLGVAPILDGNKVMVLPLREG
NPFGDKCIAWPSGTELPKEIRSDVLSWIDHSTLFQKSFVKPLAGMCESA
GMDYEEKASLDFLFG).

Any suitable T4 DNA polymerase may be used, including any T4 DNA polymerase having between 80-99.99% sequence identity to SEQ ID NO:18 and having the requisite T4 polymerase activity to facilitate NHEJ.

Any suitable MS2 sequence may be used that provides binding sites to MS2 bacteriophage coat protein. [Seminars in Virology 8, 176-185 (1997), article No. VI970120, from which the disclosure is incorporated herein by reference]. In an embodiment, a fusion protein of the disclosure comprises an MS2 sequence which comprises the sequence:

(SEQ ID NO: 20)
MASNFTQFVLVDNGGTGDVTVAPSNFANGVAEWISSNSRSQAYKVTCSV
RQSSAQKRKYTIKVEVPKVATQTVGGVELPVAAWRSYLNMELTIPIFAT
NSDCELIVKAMQGLLKDGNPIPSAIAANSGIY.

Any suitable MS2 bacteriophage coat protein sequence may be used, including any MS2 bacteriophage coat protein sequence having between 80-99.99% sequence identity to SEQ ID NO:19 and that provides requisite binding sites to MS2 RNA aptamers.

In an embodiment, the fusion protein comprises a first linker sequence that comprises the sequence SAGGGGSGGGGSGGGGSG (SEQ ID NO: 18). In an embodiment, the fusion protein comprises a second linker sequence that comprises the sequence GS.

In an embodiment, the fusion protein comprises one or more nuclear localization signals. In an embodiment, the one or more nuclear localization signals (NLSs) comprise the sequence: GPKKKRKVAAA (SEQ ID NO:21).

In an embodiment, a system of the disclosure comprises a fusion protein comprising in an N->C terminal direction a contiguous polypeptide that comprises: an MS2 protein segment, a first linker, a first NLS, a T4 DNA polymerase segment, a second linker sequence, and a second NLS. In a non-limiting embodiment, the disclosure provides a fusion protein comprising or consisting of the amino acid sequence:

(SEQ ID NO: 22)
MASNFTQFVLVDNGGTGDVTVAPSNFANGVAEWISSNSRSQAYKVTCSVRQSSAQKRKYTIKVEVPKVATQTVGGVEL
PVAAWRSYLNMELTIPIFATNSDCELIVKAMQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GSGPKKKRKVAAA,

wherein the MS2 sequence is shown in bold, the linker sequences are shown in italics, the NLS sequences are shown in enlarged font, and the T4 DNA sequence is shown in bold and italics.

Any suitable amino sequence having between 80-99.99% sequence identity to SEQ ID NO:21 wherein the sequence has the requisite T4 polymerase activity to facilitate NHEJ and that provides requisite binding sites to MS2 bacteriophage coat protein.

Any suitable nucleic acid sequence may be used in this invention that encodes SEQ ID NO:21 or the foregoing amino sequence having between 80-99.99% sequence, wherein the amino acid sequence has the requisite T4 polymerase activity to facilitate NHEJ and that provides requisite binding sites to MS2 bacteriophage coat protein.

In an embodiment, the disclosure provides a fusion protein encoded by a sequence comprising or consisting of the following nucleic acid sequence:

(SEQ ID NO: 23)
atggcttcaaactttactcagttcgtgctcgtggacaatggtgggacaggggatgtgacagtggctccttctaatttcgctaat
ggggggcagagtggatcagctccaactcacggagccaggcctacaaggtgacatgcagcgtcaggcagtctagtgcccagaaga
gaaagtataccatcaaggtggaggtccccaaagtggctacccagacagtgggcggagtcgaactgcctgtcgccgcttggaggt
cctacctgaacatggagctcactatcccaattttcgctaccaattctgactgtgaactcatcgtgaaggcaatgcaggggctcc
tcaaagacggtaatcctatcccttccgccatcgccgctaactcaggtatctacagcgctggaggaggtggaagcggaggaggag
gaagcggaggaggaggtagcggacctaagaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ggacctaagaaaaagaggaaggtg

wherein the MS2 sequence is shown in bold, the linker sequences are shown in italics, the NLS sequences are shown in enlarged font, and the T4 DNA sequence is shown in bold and italics.

A utility of the described fusion protein is the “tagging” of the T4 DNA polymerase with the MS2 protein segment. MS2 tagging is used to recruit the MS2 protein and another protein to which the MS2 is linked, such as a Cas enzyme, to RNA sequences that comprise a tetraloop and stem loop 2 of, for example, a guide RNA. These features protrude outside of a Cas9-gRNA ribonucleoprotein complex, with the distal 4 base pairs (bp) of each stem free of interactions with Cas9 amino acid side chains. The tetraloop and stem loop 2 allow the addition of protein-interacting RNA aptamers to facilitate the recruitment of effector domains to the Cas9 complex (e.g. [Nature volume 517, pages 583-588(2015)], from which the disclosure is incorporated herein by reference.

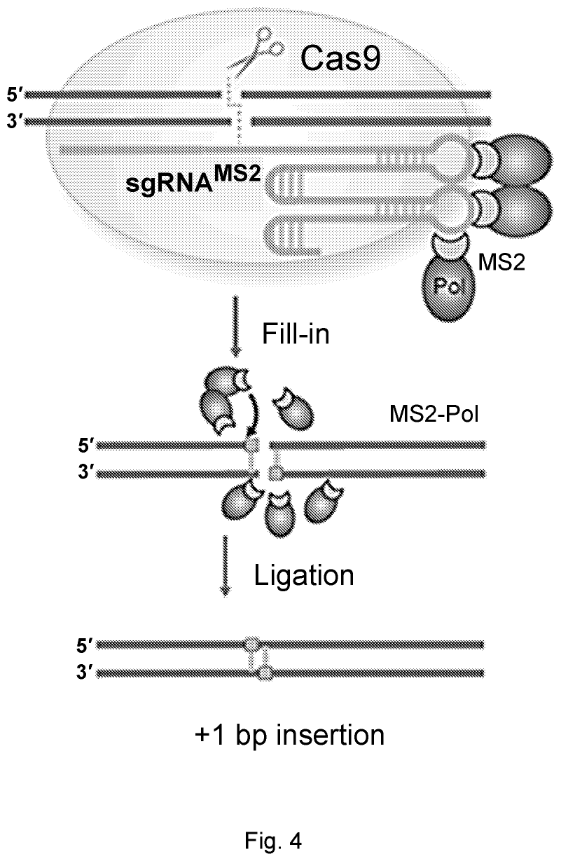

Thus, the described system is used to recruit the T4 DNA polymerase to guide RNA comprising MS2 binding domains, and a Cas enzyme. A representative illustration of this configuration is presented in FIG. 4. But other protein recruiting system may be used, such SunTag, a system for recruiting multiple protein copies to a polypeptide scaffold. [Cell. 2014 Oct. 23; 159(3): 635-646, from which the disclosure is incorporated herein by reference].

In embodiments, the T4 DNA polymerase catalyzes the synthesis of DNA in the 5′->3′ direction to create the indel after cleavage by the Cas enzyme. In embodiments, the described system inhibits microhomology-mediated end joining. In embodiments, the disclosure provides for creating a 1˜2 base pairs staggered ends with a 5′ overhang, which allow precise and predictable insertions of 1˜2 nucleotide(s) that are identical to the sequence(s) 4˜5 base pairs upstream of the PAM, by T4-mediated fill in over the staggered ends.

In specific and non-limiting embodiments, the Cas comprises a Cas9, such as Streptococcus pyogenes (SpCas9). Derivatives of Cas9 are known in the art and may also be used with the described DNA polymerase. Such derivatives may be, for example, smaller enzymes that Cas9, and/or have different proto adjacent motif (PAM) requirements. In a non-limiting embodiment, the Cas enzyme may be Cas12a, also known as Cpf1, or SpCas9-HF1, or HypaCas9, or xCas9, or Cas9-NG, or SpG, or SpRY.

In a non-limiting embodiment, the DNA endonuclease may be transposon-associated TnpB [Nature (2021).

The reference sequence of S. pyogenes is available under GenBank accession no. NC_002737, with the cas9 gene at position 854757-858863. The S. pyogenes Cas9 amino acid sequence is available under number is NP_269215. These sequences are incorporated herein by reference as they were provided on the priority date of this application or patent.

The Cas enzyme is provided with one or more suitable guide RNAs, which may be referred to as a “targeting RNA” or “targeting RNAs.” The targeting RNA is provided such that it includes suitable MS2 binding sites. In an embodiment, a suitable guide RNA comprises a sequence that is:

(SEQ ID NO: 24)
NNNNNNNNNNNNNNNNNNNNguuuuagagcuaggccaacaugaggauca
cccaugucugcagggccuagcaaguuaaaauaaggcuaguccguuauca
acuuggccaacaugaggaucacccaugucugcagggccaaguggcaccg
agucggugcuuuuuuu

wherein the bold uppercase letter represents the selected spacer, and the bold lowercase letters represent the MS2 loops to which the T4-MS2 fusion protein binds.

Any of the described components may be introduced into cells using any suitable route and form. In embodiments, the disclosure provides for use of one or more plasmids or other suitable expression vectors that encode the targeting RNA, and/or the described proteins. In embodiments, the disclosure provides RNA-protein complexes, e.g., RNAPs.

In embodiments, a viral expression vector may be used for introducing one or more of the components of the described system. Viral expression vectors may be used as naked polynucleotides, or may comprises viral particles. In embodiments, the expression vector comprises a modified viral polynucleotide, such as from an adenovirus, a herpesvirus, or a retrovirus, such as a lentiviral vector. In embodiments, one or more components of the described of CasPlus system may be delivered to cells using, for example, a recombinant adeno-associated virus (AAV) vector. Adeno-associated virus (AAV) is a replication-deficient parvovirus, the single stranded DNA genome of which is about 4.7 kb in length including 145 nucleotide inverted terminal repeat (ITRs). The nucleotide sequence of the AAV serotype 2 (AAV2) genome is presented in Ruffing el al., J Gen Virol, 75: 3385-3392 (1994). Cis-acting sequences directing viral DNA replication (rep), encapsidation/packaging and host cell chromosome integration are contained within the ITRs. As the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as an expression cassette, with the rep and cap proteins provided in trans. The sequence located between ITRs of an AAV vector genome is referred to herein as the “payload”. A recombinant AAV (rAAV) may therefore contain up to about 4.7 kb, 4.6 kb, 4.5 kb or 4.4 kb of unique payload sequence. Following infection of a target cell, protein expression and replication from the vector requires synthesis of a complementary DNA strand to form a double stranded genome. This second strand synthesis represents a rate limiting step in transgene expression. AAV vectors are commercially available, such as from TAKARA BIO® and other commercial vendors, and may be adapted for use with the described systems, given the benefit of the present disclosure. In embodiments, for producing AAV vectors, plasmid vectors may encode all or some of the well-known rep, cap and adeno-helper components. In certain embodiments, the expression vector is a self-complementary adeno-associated virus (scAAV). In scAAV vectors, the payload contains two copies of the same transgene payload in opposite orientations to one another, i.e. a first payload sequence followed by the reverse complement of that sequence. These scAAV genomes are capable of adopting either a hairpin structure, in which the complementary payload sequences hybridise intramolecularly with each other, or a double stranded complex of two genome molecules hybridised to one another. Transgene expression from such scAAVs is much more efficient than from conventional AAVs, but the effective payload capacity of the vector genome is halved because of the need for the genome to carry two complementary copies of the payload sequence. Suitable scAAV vectors are commercially available, such as from CELL BIOLABS, INC.® and can be adapted for use in the presently provided embodiments when given the benefit of this disclosure.

In this specification, the term “rAAV vector” is generally used to refer to vectors having only one copy of any given payload sequence (i.e. a rAAV vector is not an scAAV vector), and the term “AAV vector” is used to encompass both rAAV and scAAV vectors. AAV sequences in the AAV vector genomes (e.g. ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11 and AAV PHP.B. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); AAV PHP.B is described by Deverman et al., Nature Biotech. 34(2), 204-209 and its sequence deposited under GenBank Accession No. KU056473.1.

In embodiments, non-viral delivery systems may be used for introducing one or more of the components of the described system. Non-viral tools including hydrodynamic injection, electroporation and microinjection. Hydrodynamic injection can systemically deliver CasPlus into targeted tissues, including but not necessarily limited to liver. To permeate endothelial and parenchymal cells, hydrodynamic injections require a high injection volume, speed and pressure that limit central nervous system therapies. Electroporation and microinjection can be used for germline editing or embryo manipulation. Chemical vectors, such as lipids and nanoparticles, are widely used for delivery. Cationic lipids interact with negatively charged DNA and the cell membrane, protecting the DNA and cellular endocytosis. DNA nanoparticles, such as, are potential delivery strategies. DNA conjugated to gold nanoparticles (CRISPR-gold) complexed with cationic endosomal disruptive polymers can deliver CasPlus into animal cells.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference. Further, any of a variety of therapeutic delivery agents can be used, and include but are not limited to nanoparticles, lipid nanoparticle (LNP), fusosomes, exosomes, and the like. In embodiments, a biodegradable material can be used. In embodiments, poly(lactide-co-galactide) (PLGA) is a representative biodegradable material, but it is expected that any biodegradable material, including but not necessarily limited to biodegradable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In embodiments, a combination of proteins, and a combination one or more proteins and polynucleotides described herein, may be first assembled in vitro and then administered to a cell or an organism.

The cells into which the described systems are introduced are not particularly limited, and may include postmitotic adult tissues, which are considered to be refractory to HDR, such as for example, heart and skeletal cells. The disclosure is not necessarily limited to such cells, and may also be used with, for example, with totipotent, pluripotent, multipotent, or oligopotent stem cells. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are muscle precursor cells, such as quiescent satellite cells, or myoblasts, including but not necessarily limited to skeletal myoblasts and cardiac myoblasts. In embodiments, the disclosure includes obtaining cells from an individual, modifying the cells ex vivo using a system as described herein, and reintroducing the cells or their progeny into the individual or an immunologically matched individual for prophylaxis and/or therapy of a condition, disease or disorder, as described above. In embodiments, the cells modified ex vivo as described herein are autologous cells. In embodiments, the cells are mammalian cells. The disclosure is thus suitable for a wide range of human, veterinary, experimental animal, and cell culture uses.

The following Examples are intended to illustrate but not limit the disclosure.

### Example 1

CRISPR/Cas9-Guided T4 DNA Polymerase Facilitates the Generation of Insertions Via Filling in the Staggered DNA with 5′ Overhang.

Analysis of the mutational profiles generated from the repair of CRISPR/Cas9 mediated DNA double-stranded breaks via Non-homology end joining (NHEJ) revealed that CRISPR/Cas9 permits the production of precise, reproductive and predictable indels on the basis of sequence context flanking the cut site, as well as the generation of undesirable large deletions extending over many kilobases¹⁻⁴. In general, most DSBs created by Cas9 are blunt ends, which undergo end processing and lead to the production of deletions. In some cases, Cas9 enables the generation of 1˜2 base pairs staggered ends with 5′ overhang, which allow precise and predictable insertions of 1˜2 nucleotide(s) that are identical to the sequence(s) 4˜5 base pairs upstream of the PAM without template donor (FIG. 1A). Cas9-mediated insertions are resultant from the filling-in of the overhang by certain DNA polymerase before ligation5,6. DNA polymerase lambda and mu, whose defects are usually associated with large deletions in the vicinity of induced DSBs, are two essential proteins involved in filling in the maps generated in the process of repairing DSBs via NHEJ in mammalian cells⁷. We analyzed whether the local recruitment of a DNA polymerase by an engineered CRISPR/Cas9 system could fill in the staggered DNA ends before that being processed by endonucleases, thus facilitating the generation of insertions. To explore this possibility, we established a 293T reporter cell line which stably incorporated with a tdTomato gene with 151A deletion and designed a 20-nt gRNA (termed as tdTomato-sgRNA) that has a strong bias to re-insert an A at position 151 on the basis of the sequence (FIG. 1). Next, MS2-tagged DNA polymerase lambda, DNA polymerase Mu, DNA polymerase Beta, yeast derived DNA polymerase 4, bacteria derived DNA polymerase I or Klenow fragment (KF), or bacteriophage derived T4 DNA polymerase (without the 5′-3′ exonuclease activity) and plasmids expressing CRISPR/Cas9 and tdTomato-sgRNA were respectively transfected into 293T reporter cells. PCR products harboring approximate 150 bp upstream and downstream of target site were amplified and sequenced from tdTomato⁺/GFP⁺ or tdTomato⁻/GFP⁺ cell populations. Analysis of the Sanger sequencing results revealed that, in tdTomato⁺/GFP⁺ populations, no obvious indels profiles change among all the treatments, whereas in tdTomato⁻/GFP⁺ populations, the insertion of 2-bp was significantly increased in T4 DNA polymerase-transfected cells relative to other treatments (FIGS. 1C-1E). High-throughput results further confirmed that the overall 2-bp insertions among all the indels was increased up to 35% in cells with T4 DNA polymerase compared to 2% detected in control cells (FIG. 1F). Analysis of the pattern of insertions revealed that the majority of 1 or 2 nucleotides respectively inserted around the target site are not random but template-dependent (FIG. 1G). Next, we validated the effect of T4 DNA polymerase on three endogenous target sites that enable the production of 1˜2-bp insertions (FIG. 1H). All altogether, these results indicate CRISPR/Cas9-mediated T4 DNA polymerase facilitates the generation of insertions via filling in the staggered DNA with 5′ overhangs.

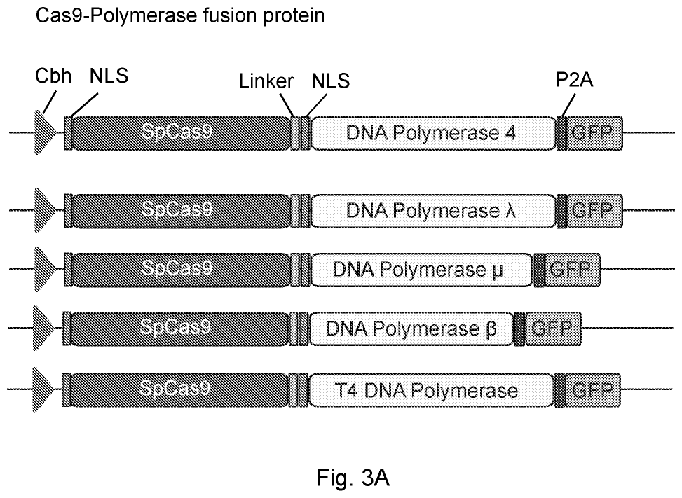

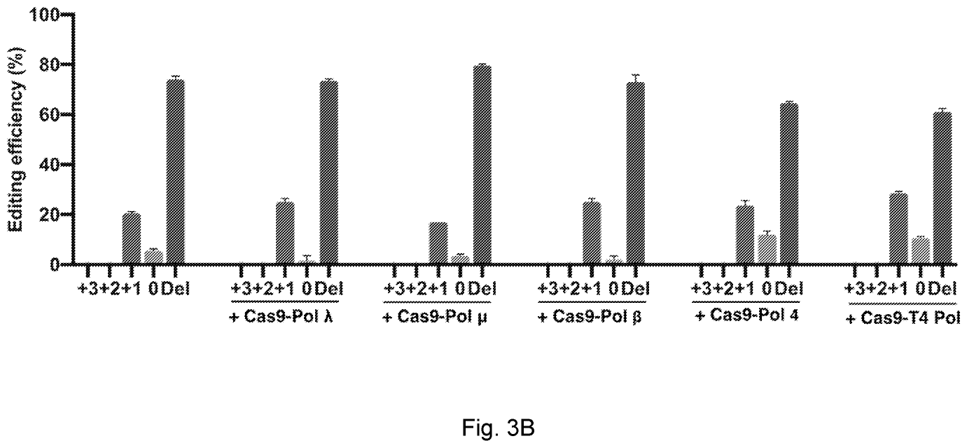

To investigate whether fusion of DNA polymerase to the carboxyl terminal of SpCas9 via a flexible link promotes the production of insertions, we transfected Cas9-DNA polymerase fusion vectors into 293T tdTomato reporter cells. However, unlike ms2-tagged T4 DNA polymerase, Cas9-fused T4 DNA polymerase was unable to enhance insertions (FIGS. 3A-3B).

### Example 2

CRISPR/Cas9-Guided T4 DNA Polymerase Impairs MMEJ Repair Pathway.

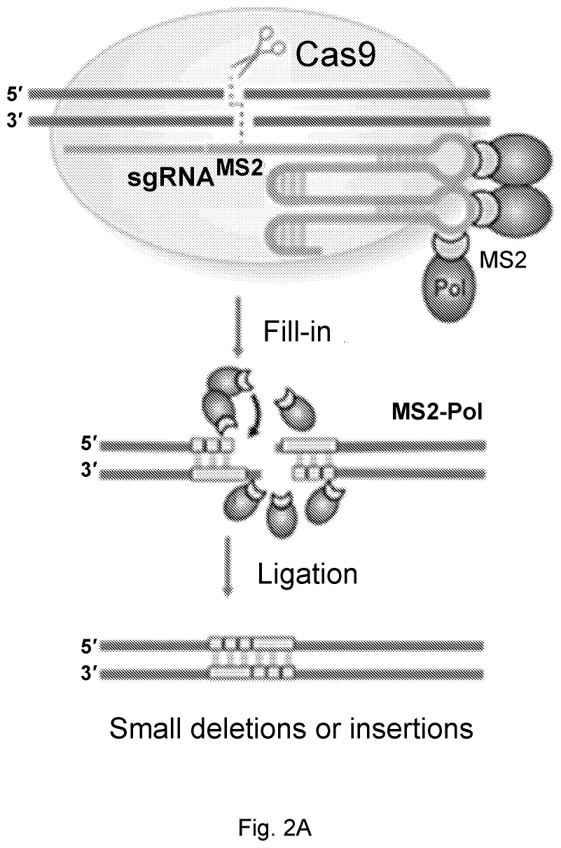

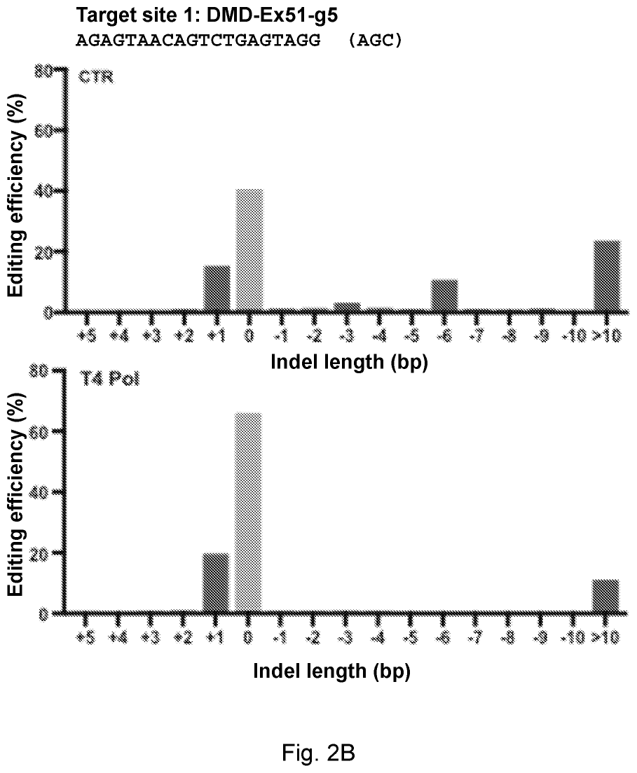

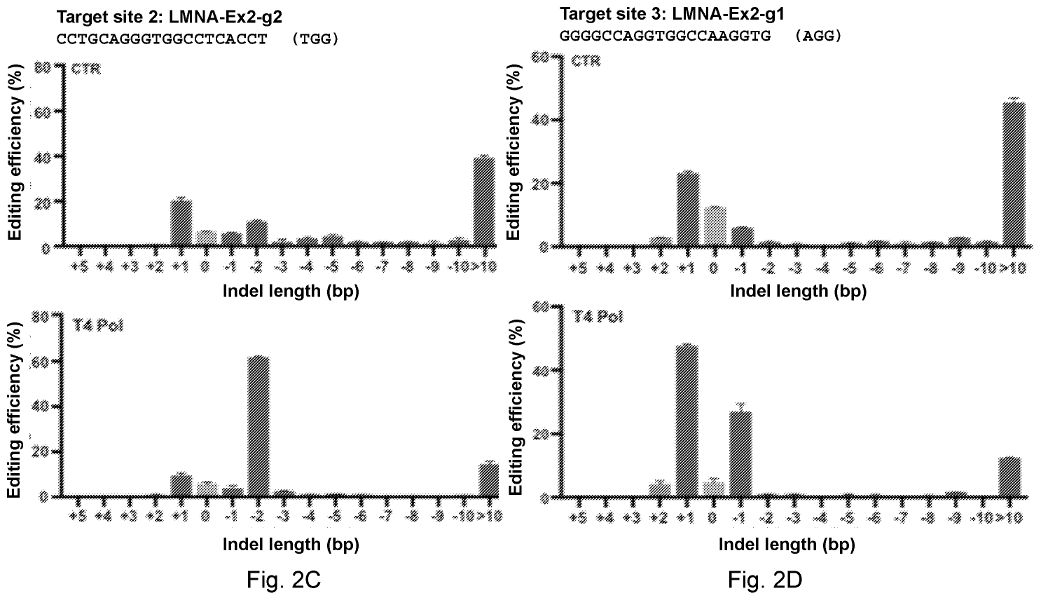

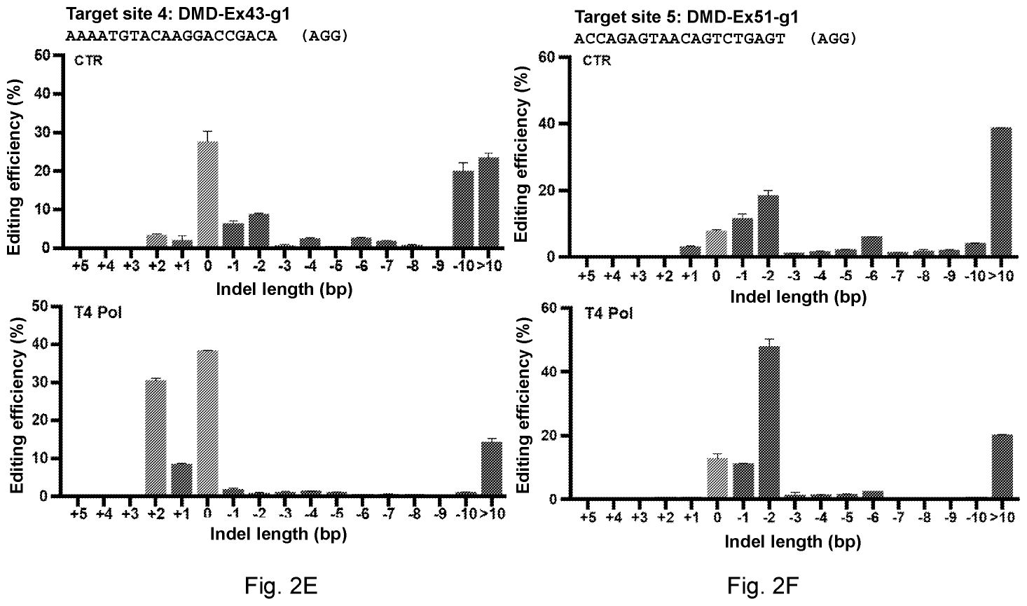

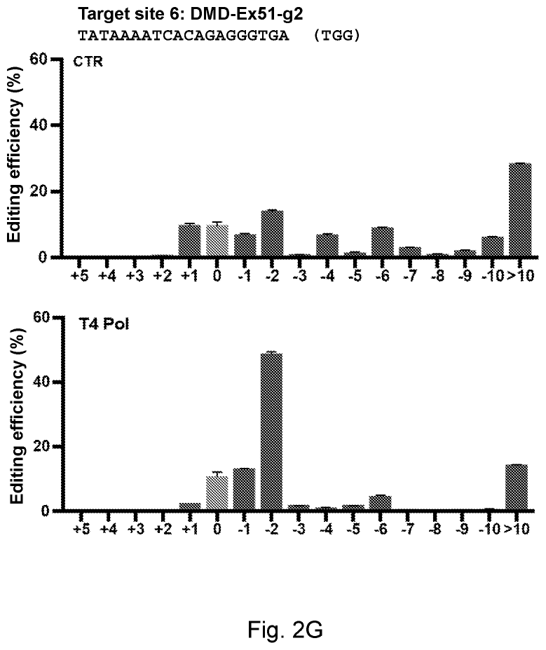

Microhomology-mediated end joining, also called alternative end joining, is a DNA damage response occurring following DNA DSBs. MMEJ is an alternative repair pathway to HDR, initiated following DNA end resection. Based on a sufficient region of sequence homology flanking a DSB, approximately 5-25 bp, a DSB is repaired through annealing the homologous regions together, thereby deleting one repeat and the intermediate sequence. Microduplications and sequence repeats are a common DNA replication error resulting in nascent genetic disease. Inducing targeted DSB at a site flanked by these repeats meets the criteria to initiate the MMEJ DNA damage response, thereby having the potential to revert pathogenic microduplications and sequence repeats into a wild-type allele. The repair outcomes of CRISPR/Cas9 induced double-strand breaks (DSBs) via MMEJ pathway enable precise and predictable deletions of the microhomology sequences and the intervening region, which was harnessed to correct pathogenic mutations caused by microduplication⁸. High-throughput assay of Cas9-induced DNA repair products show that half of the indels detected are microhomology-mediated deletions. Inhibitors of poly (ADP-ribose) polymerase 1 (PARP-1) suppress the DNA repair via MMEJ, thus leading to fewer microhomology-dependent deletions. In principle, if T4 DNA polymerase enables the filling-in of SpCas9-induced staggered DNA ends with 5′ overhangs before that being trimmed by endonucleases, we proposed that it also enables increasing the fill-in efficiency and prevents relative long-term DNA resection, thus impairing MMEJ repair and permitting the generation of smaller indels products (FIG. 2A). To confirm this potentiality, we tested the ability of T4 DNA polymerase in disrupting MMEJ repair pathway in six target sites mainly dependent on MMEJ for DNA repair. High-throughput results showed that most of the relatively big deletions (greater than 10 bp) either created in a MH-dependent or MH-independent repair pathway across six different sites were substantially decreased by T4 DNA polymerase in the meanwhile products with 1-2 bp indels were significantly increased. Together, these results indicate CRISPR/Cas9-guided T4 DNA polymerase impairs MMEJ repair pathway and enables to convert the MH-dependent or MH-independent big deletions into smaller products with 1˜2-bp indels.

Representative guide RNA sequences used to develop data presented in this disclosure are as follows, with the respective PAM sequences indicated in the right column:

| | Name | gRNA sequence | PAM | SEQ ID NO |
|---|---|---|---|---|
| Target site 1 | DMD-Ex51-g5 | AGAGUAACAGUCUGAGUAGG | AGC | 25 |
| Target site 2 | LMNA-g2 | CCUGCAGGGUGGCCUCACCU | TGG | 26 |
| Target site 3 | LMNA-g1 | GGGGCCAGGUGGCCAAGGUG | AGG | 27 |
| Target site 4 | DMD-Ex43-g1 | AAAAUGUACAAGGACCGACA | AGG | 28 |
| Target site 5 | DMD-Ex51-g1 | ACCAGAGUAACAGUCUGAGU | AGG | 29 |
| Target site 6 | DMD-Ex51-g2 | UAUAAAAUCACAGAGGGUGA | TGG | 30 |
| Target site 7 | tdTomato-sgRNA | CAAGCUGAAGGUGACCAGGG | CGG | 31 |
| Target site 8 | Mybpc3-323-g3 | AUUUAUAGCCCAAGAUUUCC | TGG | 32 |
| Target site 9 | LMNA-Ex3-g2 | GCCUGCUUCCUCACAGCUUG | AGG | 33 |
| Target site 10 | Mybpc3-323-g2 | UUCUUGAACCAGGAAAUCUU | GGG | 34 |

The following reference listing is not an indication that any reference is material to patentability.
1. Shen, M. W. et al. Predictable and precise template-free CRISPR editing of pathogenic variants. Nature 563, 646-651 (2018).
2. Kosicki, M., Tomberg, K. & Bradley, A. Repair of double-strand breaks induced by CRISPR-Cas9 leads to large deletions and complex rearrangements. Nat Biotechnol 36, 765-771 (2018).
3. Shin, H. Y. et al. CRISPR/Cas9 targeting events cause complex deletions and insertions at 17 sites in the mouse genome. Nat Commun 8, 15464 (2017).
4. Allen, F. et al. Predicting the mutations generated by repair of Cas9-induced double-strand breaks. Nat Biotechnol (2018).
5. Shi, X. et al. Cas9 has no exonuclease activity resulting in staggered cleavage with overhangs and predictable di- and tri-nucleotide CRISPR insertions without template donor. Cell Discov 5, 53 (2019).
6. Shou, J., Li, J., Liu, Y. & Wu, Q. Precise and Predictable CRISPR Chromosomal Rearrangements Reveal Principles of Cas9-Mediated Nucleotide Insertion. Mol Cell 71, 498-509 e494 (2018).
7. Capp, J. P. et al. The DNA polymerase lambda is required for the repair of non-compatible DNA double strand breaks by NHEJ in mammalian cells. Nucleic Acids Res 34, 2998-3007 (2006).
8. Iyer, S. et al. Precise therapeutic gene correction by a simple nuclease-induced double-stranded break. Nature 568, 561-565 (2019).

1. A fusion protein comprising a T4 DNA polymerase segment and a segment of an MS2 bacteriophage coat protein.

2. The fusion protein of claim 1, further comprising at least one nuclear localization signal.

3. The fusion protein of claim 2, wherein the T4 DNA polymerase segment and the segment of the MS2 protein are separated by a first linker sequence.

4. The fusion protein of claim 3, further comprising the first linker amino acid sequence that links the MS2 segment to a first nuclear localization signal, and a second linker sequence that links the T4 DNA polymerase segment to a second nuclear localization signal.

5. A complex comprising a double stranded DNA template, a Cas enzyme, a guide RNA comprising MS2 bacteriophage coat protein binding sites, a protein comprising a T4 DNA polymerase, and an MS2 binding protein.

6. The complex of claim 5, further comprising a guide RNA comprising MS2 protein binding sequences.

7. The complex of claim 5, wherein the Cas enzyme is Cas9.

8. A cell comprising a complex of claim 5.

9. A pharmaceutical formulation comprising a fusion protein of claim 1.

10. A method for producing an indel at a selected chromosome locus in a cell, the method comprising introducing into the cell a fusion protein of claim 1, a Cas enzyme, and a guide RNA comprising MS2 protein binding sites, such that the T4 DNA polymerase and the MS2 binding protein, the Cas enzyme, and the guide RNA produce the indel at the selected chromosome locus.

11. The method of claim 10, wherein the indel corrects a mutation in an open reading frame encoded by the selected chromosome locus.

12. The method of claim 11, wherein the selected chromosome locus comprises a mutation in a gene that is correlated with a monogenic disease.

13. The method of claim 12, wherein the monogenic disease is muscular dystrophy, and wherein the gene encodes a mutated dystrophin protein.

14. The method of claim 13, wherein the indel corrects the gene encoding the mutated dystrophin protein.

15. The method of claim 14, wherein the indel comprises a one or two base pair insertion.

16. A kit comprising a fusion protein of claim 1, or an expression vector encoding said fusion protein.

17. The kit of claim 16, further comprising a Cas enzyme or an expression vector encoding a Cas enzyme.

18. The kit of claim 17, further comprising a guide RNA or an expression vector encoding said guide RNA, wherein the guide RNA comprises MS2 protein binding sequences, and wherein the guide RNA comprises a sequence targeted to a selected chromosome locus.

19. An expression vector encoding a fusion protein of claim 1.

20. A cDNA encoding a fusion protein of claim 1.


SEQUENCE LISTING (34 sequences)

SEQ ID NO: 1 - DNA, 23 bp, artificial sequence
  CRISPR related sequence
caagctgaaggtgaccagggcgg

SEQ ID NO: 2 - DNA, 24 bp, artificial sequence
  CRISPR related sequence
caagctgaaggtgaccaagggcgg

SEQ ID NO: 3 - DNA, 20 bp, artificial sequence
  CRISPR related sequence
caagctgaaggtgaccaggg

SEQ ID NO: 4 - DNA, 22 bp, artificial sequence
  CRISPR related sequence
gttcgattccactggtcccgcc

SEQ ID NO: 5 - DNA, 20 bp, artificial sequence
  CRISPR related sequence
atttatagcccaagatttcc

SEQ ID NO: 6 - DNA, 20 bp, artificial sequence
  CRISPR related sequence
gcctgcttcctcacagcttg

SEQ ID NO: 7 - DNA, 20 bp, artificial sequence
  CRISPR related sequence
ttcttgaaccaggaaatctt

SEQ ID NO: 8 - DNA, 20 bp, artificial sequence
  CRISPR related sequence
agagtaacagtctgagtagg

SEQ ID NO: 9 - DNA, 20 bp, artificial sequence
  CRISPR related sequence
cctgcagggtggcctcacct

SEQ ID NO: 10 - DNA, 20 bp, artificial sequence
  CRISPR related sequence
ggggccaggtggccaaggtg

SEQ ID NO: 11 - DNA, 20 bp, artificial sequence
  CRISPR related sequence
aaaatgtacaaggaccgaca

SEQ ID NO: 12 - DNA, 20 bp, artificial sequence
  CRISPR related sequence
accagagtaacagtctgagt

SEQ ID NO: 13 - DNA, 20 bp, artificial sequence
  CRISPR related sequence
tataaaatcacagagggtga

SEQ ID NO: 14 - protein, 18 aa, artificial sequence
  self cleaving peptide sequence
EGRGSLLTCGDVEENPGP

SEQ ID NO: 15 - protein, 19 aa, artificial sequence
  self cleaving peptide sequence
ATNFSLLKQAGDVEENPGP

SEQ ID NO: 16 - protein, 20 aa, artificial sequence
  self cleaving peptide sequence
QCTNYALLKLAGDVESNPGP

SEQ ID NO: 17 - protein, 22 aa, artificial sequence
  self cleaving peptide sequence
VKQTLNFDLLKLAGDVESNPGP

SEQ ID NO: 18 - protein, 18 aa, artificial sequence
  linker
SAGGGGSGGGGSGGGGSG

SEQ ID NO: 19 - protein, 897 aa, Bacteriophage T4
KEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 20 - protein, 130 aa, artificial sequence
  MS2 binding protein
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 21 - protein, 11 aa, artificial sequence
  nuclear localization signal
GPKKKRKVAAA

SEQ ID NO: 22 - protein, 1065 aa, artificial sequence
  fusion protein
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGGSGPKKKRKVAAA

SEQ ID NO: 23 - DNA, 3186 bp, artificial sequence
  cDNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgaaggaattctacatcagcatcgagaccgtgggtaacaacatcgtggaaagatatattgacgaaaacggcaaggagagaaccagagaggtggaatacctgcctacaatgttccggcactgtaaagaggaatccaagtacaaggatatctacggcaaaaactgcgcccctcagaaattccccagcatgaaagacgccagagattggatgaagagaatggaggatatcggactggaagccctgggcatgaacgatttcaagctggcctacatctccgatacatacggaagcgagatcgtgtatgatagaaaattcgtgcgggtggccaattgtgacattgaggtgaccggcgacaagttccctgatcccatgaaagctgaatatgagatcgacgccattacccactacgacagcatcgacgacagattctacgtgttcgacctgctgaactccatgtacggcagcgtgtccaagtgggacgctaagctggccgccaagctggactgcgagggcggcgacgaggttccacaagagatcctggaccgggtcatctacatgcccttcgacaacgagagggacatgctgatggaatacatcaacctgtgggagcagaagcgccccgccatttttacaggctggaacatcgagggcttcgacgtgccttatatcatgaatagagtgaaaatgatcctgggagaacggagcatgaaaagattcagccctatcggcagagtgaagagcaagctgatccaaaacatgtacggctccaaggaaatctatagcatcgatggcgtgtccatcctggattacctggacctgtacaaaaagttcgccttcaccaacctgccatctttctctcttgagagcgtcgcccagcacgagacaaagaagggcaagctgccgtacgacggtcctatcaacaagctgagagaaacaaatcaccagagatacatcagctacaacatcatcgatgtggaaagcgttcaggccatcgataaaatcagaggcttcatcgacctggtgctgtctatgtcttactacgccaagatgccttttagcggagtgatgagccctatcaagacctgggatgccatcatcttcaacagcctgaagggcgaacacaaggtgatcccccaacagggcagccacgtgaagcagagcttcccaggcgcttttgtgttcgagcccaagcccatagcgcggagatacatcatgagctttgatctgaccagcctgtaccccagcatcattcggcaagtgaacatttctccagaaaccatcagaggccagtttaaggtgcaccctatccacgagtatattgcaggcaccgctcctaaacctagcgacgagtacagctgctctcctaacggctggatgtacgacaagcaccaggagggaatcatccctaaggaaattgccaaggtgtttttccagcggaaggactggaagaaaaaaatgttcgccgaggaaatgaacgccgaggccatcaagaagatcatcatgaagggcgccggcagctgctccaccaagcctgaggtggaaagatacgtgaagttcagcgacgatttcctgaatgagctcagcaactacaccgagtctgtcctgaactcactgattgaggaatgcgagaaggccgccaccctggctaataccaaccagctgaaccggaagattctgatcaacagcctgtacggagctctgggcaatattcacttcagatactacgatctgcgaaacgccacagctattacaattttcggccaggtgggcatccagtggatcgccagaaagatcaatgagtacctgaacaaggtgtgcggcaccaacgacgaggacttcatcgccgctggcgatactgatagcgtgtacgtttgtgtggacaaggtcatcgagaaggttggcctggacagatttaaggaacagaacgacctcgtggagttcatgaaccagttcggaaagaagaagatggaacccatgatcgatgtggcttatagagagctgtgcgactacatgaacaacagagagcacctgatgcacatggatagagaagctatttcttgccctcctctgggctctaagggagtgggcggattttggaaagccaaaaagagatacgccctgaatgtgtacgacatggaagataagagattcgccgagcctcacctgaaaatcatgggcatggaaacacagcagagcagcacccctaaggctgtgcaggaggccctggaagagtctatccggagaatcttgcaggagggcgaggaaagcgtgcaggagtactacaagaacttcgagaaagaatacagacagctggactacaaggtgatcgcggaggtgaagaccgctaatgatatcgccaagtacgacgacaagggctggcccggcttcaagtgccccttccacatcagaggcgtgctcacctaccgcagagccgtttccggcctgggcgtggcccctatcctggatggaaacaaagtcatggtgctgcctctgagagagggcaacccctttggagataaatgcatcgcttggcctagcggcactgagctgcccaaggaaatccgctccgacgtgctgagctggatcgatcacagcaccctgttccaaaagtccttcgtgaagcccctggccggcatgtgcgagtccgccggcatggactacgaggaaaaggccagcctggatttcctgttcggcggatccggacctaagaaaaagaggaaggtg

SEQ ID NO: 24 - RNA, 163 nt, artificial sequence
  MS2 binding sequence
  misc_feature (1)..(20): n is a, c, g, or u
nnnnnnnnnnnnnnnnnnnnguuuuagagcuaggccaacaugaggaucacccaugucugcagggccuagcaaguuaaaauaaggcuaguccguuaucaacuuggccaacaugaggaucacccaugucugcagggccaaguggcaccgagucggugcuuuuuuu

SEQ ID NO: 25 - RNA, 20 nt, artificial sequence
  guide RNA
agaguaacagucugaguagg

SEQ ID NO: 26 - RNA, 20 nt, artificial sequence
  guide RNA
ccugcaggguggccucaccu

SEQ ID NO: 27 - RNA, 20 nt, artificial sequence
  guide RNA
ggggccagguggccaaggug

SEQ ID NO: 28 - RNA, 20 nt, artificial sequence
  guide RNA
aaaauguacaaggaccgaca

SEQ ID NO: 29 - RNA, 20 nt, artificial sequence
  guide RNA
accagaguaacagucugagu

SEQ ID NO: 30 - RNA, 20 nt, artificial sequence
  guide RNA
uauaaaaucacagaggguga

SEQ ID NO: 31 - RNA, 20 nt, artificial sequence
  guide RNA
caagcugaaggugaccaggg

SEQ ID NO: 32 - RNA, 20 nt, artificial sequence
  guide RNA
auuuauagcccaagauuucc

SEQ ID NO: 33 - RNA, 20 nt, artificial sequence
  guide RNA
gccugcuuccucacagcuug

SEQ ID NO: 34 - RNA, 20 nt, artificial sequence
  guide RNA
uucuugaaccaggaaaucuu